(19) United States
(12) Patent Application Publication
(10) Pub. No.: US 2011/0021962 A1
(43) Pub. Date: Jan. 27, 2011

(54) SHOULDER BRACE

(75) Inventor: Mark Sorrenti, Sydney (AU)

Correspondence Address: FOLEY AND LARDNER LLP;SUITE 500, 3000 K STREET NW, WASHINGTON, DC, 20007, US

(73) Assignee: S.A.M. Bracing PTY Ltd.

(21) Appl. No.: 12/809,536

(22) PCT Filed: Dec. 19, 2008
(86) PCT No.: PCT/AU08/01883

§ 371 Date: Sep. 28, 2010

Related U.S. Application Data
(60) Provisional application No. 61/079,315, filed on Jul. 9, 2008.

(30) Foreign Application Priority Data
Dec. 19, 2007 (AU) 2007906967

(51) Int. Cl. A61F 5/00

(52) U.S. Cl. 602/20

(57) ABSTRACT

A shoulder brace for supporting a person's injured or post-operated shoulder, the brace comprising: a support for the person to wear; and an arm connected to the support, the arm being adapted for supporting the person's arm in a raised position, wherein the arm is movable with respect to the support and is resiliently biased against downward movement with respect to the support. A method of treating a person's injured or post-operated shoulder, the method comprising the steps of: (a) wearing the shoulder brace described above; and (b) having the person use their arm, which has the injured or post-operated shoulder, to apply a force against the resilient bias of the arm of the shoulder brace.

### TECHNICAL FIELD

The disclosure relates to a shoulder brace.

### BACKGROUND

Braces are used to support limbs and joints after injury or surgery. The braces are used to severely limit, if not prevent, movement of the limb or joint so as to reduce strain on the surrounding muscles, ligaments and tendons and thereby aid the healing process. For example, shoulder braces are used to support a person's arm in a raised position as treatment for weakness in or injury of the person's shoulder. However, immobilising limbs and joints in this way can lead to muscle weakness which may necessitate further rehabilitation of the limbs and joints once the brace is removed.

### SUMMARY OF THE DISCLOSURE

According to a first aspect, there is provided a shoulder brace for supporting a person's injured or post-operated shoulder comprising:

a support for the person to wear; and

an arm connected to the support, the arm being adapted for supporting the person's arm in a raised position, wherein the arm is movable with respect to the support and is resiliently biased against downward movement with respect to the support.

The brace arm can generally be movable under the weight of and/or by applied force from the person's arm supported by the brace arm.

In an embodiment, the arm comprises first and second portions to enable adjustment of the length of the arm.

This allows use of a brace by a range of people with different length arms.

In an embodiment, the support comprises at least one plate. The at least one plate can be shaped to fit comfortably against a person's torso. The plate may be made of a flexible material for enabling it to be adjusted to the shape of the person's torso. The plate may be fixed to the support.

In a further embodiment, the support comprises at least two plates and a stem interconnecting the at least two plates.

In an embodiment, the shoulder brace comprises a resilient joint between the arm and the stem. The resilient joint may comprise a resistor which resists movement of the arm. The resistance of the resistor may be adjustable.

In an embodiment, the resilient joint can be removably replaced with a joint of differing resistance. This replacement can allow for resistance to be varied to enhance/promote shoulder rehabilitation.

In an embodiment, the resilient joint comprises a first member which, in use, sits in a generally static location with respect to a person's shoulder, and a second member pivotally mounted to the first member, the second member being adapted for coupling to the arm. The resistor may be a spring located for operation between the first and second members. The spring can take the form of a torsion spring, a compression spring, a piston-like spring etc.

In an embodiment, the resilient joint comprises spaced, opposing joints located, in use, anteriorly and posteriorly with respect to a user's shoulder (i.e. two joints, one located in an anterior position, and the other located in a posterior position).

In an embodiment, the first member is connected to the stem by a first mounting bracket, and the second member is connected to the arm by a second mounting bracket. The second mounting bracket may be shaped to support (e.g. underlie) the person's arm in use.

In one embodiment, the arm further comprises a cradle located for cradling the person's forearm in use.

In one embodiment, the shoulder brace further comprises an extension support attachable to the cradle, for supporting the person's wrist in use.

In an embodiment, both the cradle and extension support are ergonomically shaped to comfortably fit the person's forearm. The cradle may be made of a flexible material which can be readily shaped to the person's forearm.

In an embodiment, the cradle is connected to the arm by a ball and socket joint. The ball and socket joint enables some limited, yet controlled, movement of the person's arm.

In an embodiment, the socket of the ball and socket joint comprises a cup and a U-shaped clip. The U-shaped clip can fit over the ball when it is positioned in the cup. The U-shaped clip generally prevents the ball lifting out of the cup and the joint from being broken.

In an embodiment, the cradle also comprises at least one, preferably two, forearm straps for keeping the person's arm strapped in the cradle. The forearm straps may be connected to the cradle.

In one embodiment, the support further comprises at least two straps for attaching the support around and to the person's body. The first strap may be attached over the person's non-injured shoulder, and subsequent strap(s) can be attached around the person's torso.

In one embodiment, the support further comprises a vest portion to be worn over the person's torso, adjacent to the shoulder being supported by the shoulder brace.

In one embodiment, the vest portion is made of a soft material which can be readily adapted to the shape of the person's torso.

In one embodiment, the at least one plate is connected to the vest portion. The at least two straps may also be connected to the vest portion.

According to a second aspect, there is provided a method of treating a person's injured or post-operated shoulder, the method comprising the steps of:
(a) wearing the shoulder brace according to the first aspect; and
(b) having the person use their arm, which has the injured or post-operated shoulder, to apply a force against the resilient bias of the arm of the shoulder brace.

In an embodiment, the person continuously applies a force against the resilient bias of the shoulder brace's arm during treatment.

In another embodiment, the person periodically applies a force against the resilient bias of the shoulder brace's arm during treatment.

In an embodiment, the method further comprises the step of increasing the strength of the resilient bias of the arm of the shoulder brace.

In an embodiment, the step of increasing the strength of the resilient bias occurs in stepwise increases over the length of treatment.

In an embodiment, the step of increasing the strength of the resilient bias comprises replacing the resistor which resists movement of the arm with a resistor of greater resistance.

In an embodiment, replacing the resistor comprises replacing the spring inside the resilient joint.

In an embodiment, the method of treatment occurs for approximately 12 to 18 weeks.

### BRIEF DESCRIPTION OF THE DRAWINGS

Embodiments of the shoulder brace and method of use will now be described, by way of example only, with reference to the accompanying drawings, in which:

FIG. 1 is a front perspective view of a shoulder brace, at least in part, according to an embodiment;

FIG. 2 is a side perspective view of the shoulder brace of FIG. 1;

FIG. 3 is an enlarged view of the resilient joint shown in FIG. 1;

FIG. 4 is a front perspective view of a shoulder brace, at least in part, according to an alternate embodiment;

FIG. 5 is a side perspective view of the shoulder brace of FIG. 4;

FIG. 6 is a front perspective view of a shoulder brace, at least in part, according to an alternate embodiment;

FIG. 7 is an enlarged view of the resilient joint shown in FIG. 6;

FIG. 8 is a front perspective view of a shoulder brace, at least in part, according to an alternate embodiment;

FIG. 9 is an exploded front perspective view of the shoulder brace of FIG. 8; and

FIG. 10 is a front view of a shoulder brace according to an embodiment being worn by a mannequin.

### DETAILED DESCRIPTION OF SPECIFIC EMBODIMENTS

Referring to the Figures, a shoulder brace 10 for supporting a person's injured or post-operated shoulder is shown. The shoulder brace 10 comprises a support 11 for the person to wear and an arm 12 connected to the support 11 for supporting the person's arm in a raised position. The arm 12 is movable with respect to the support 11 and is resiliently biased against downward movement relative to the support 11.

The arm 12 is movable under the weight of and/or applied force by the person's arm supported by the arm 12. Movement of the arm 12, in the embodiment shown in the Figures, involves rotation of the arm 12 so that a portion of the arm 12 moves in a downward direction, against the resilient bias. The shoulder brace 10 may be worn for 12-18 weeks, depending on the severity of the injury or surgery, and the rate of healing.

By the person using their arm to move against the resilient bias of the arm 12 of the shoulder brace 10 during this time, this assists muscle, ligament and tendon strengthening in the person's shoulder and back, as well as promoting collagen growth in and around the shoulder joint. This “resistance training” of the person's shoulder using the shoulder brace 10 greatly enhances rehabilitation of the shoulder after injury or surgery, reducing treatment time and providing better preparation for post-operative physiotherapy.

The arm 12 has an upper limited position past which the arm 12 cannot move. At this upper limited position, the arm 12 supports the person's arm (and thus shoulder) at an appropriate height. Limitation of movement of the arm 12 past this point prevents over-rotation of the person's shoulder and hence further damage to the shoulder joint.

The arm 12 has first and second ends 13, 14, the first end 13 located proximate to the support 11 and the second end 14 located furthest away from the support 11. The arm 12 is rotatable about the first end 13. The arm 12 comprises first and second portions 15 and 16, respectively. The first and second portions 15, 16 are arranged to enable adjustment of the length of the arm 12 for each person using the shoulder brace 10.

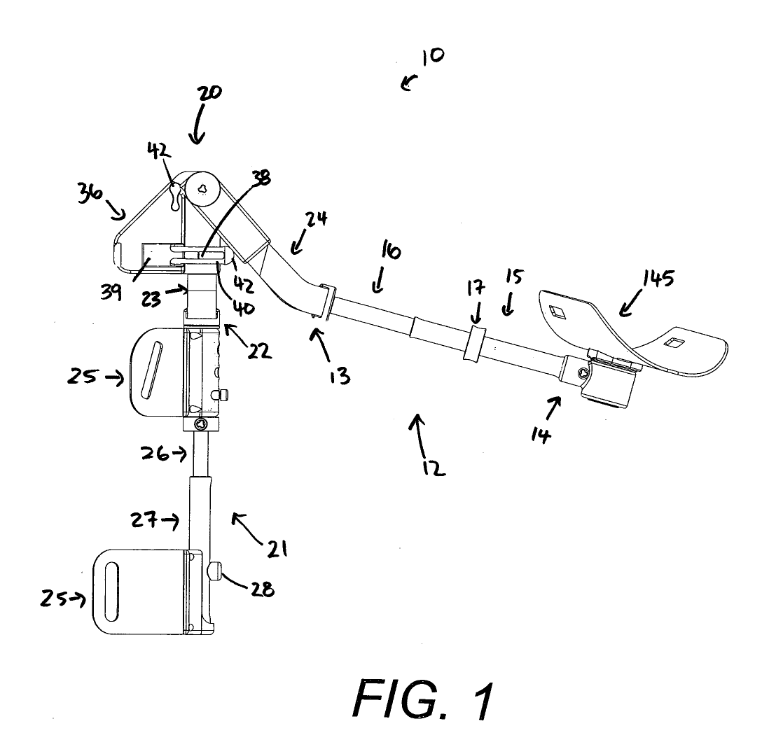

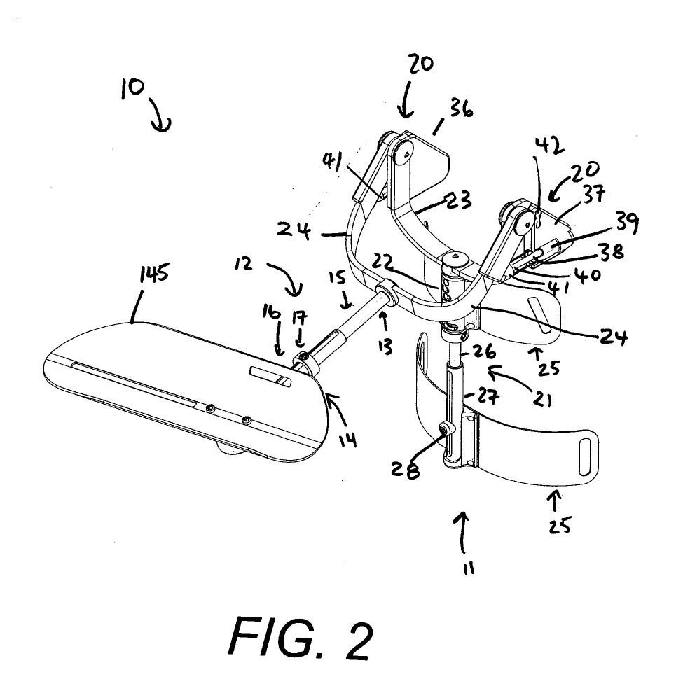

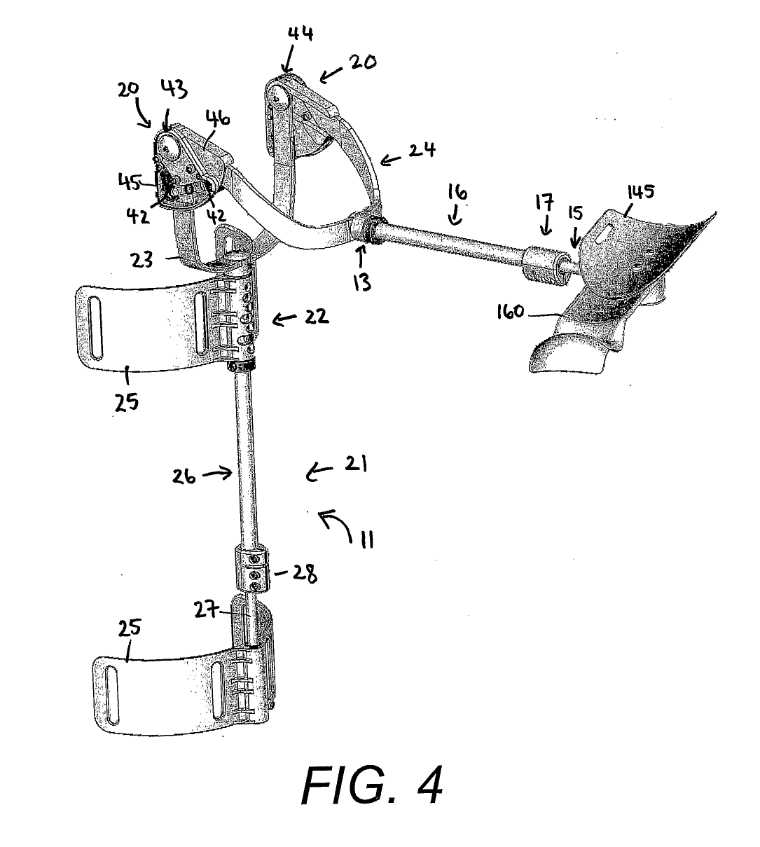

In FIGS. 4, 5, 8 and 9 the first portion 15 is sized to fit inside the second portion 16 (although in other embodiments, as shown in FIGS. 1 and 2, the first portion 15 can be sized to fit around the second portion 16). In FIGS. 1, 2, 4 and 5, the first and second portions are telescopic, and are slidable with respect to each other. A locking mechanism 17 is used to lock the first and second portions 15, 16 at the length required for an individual.

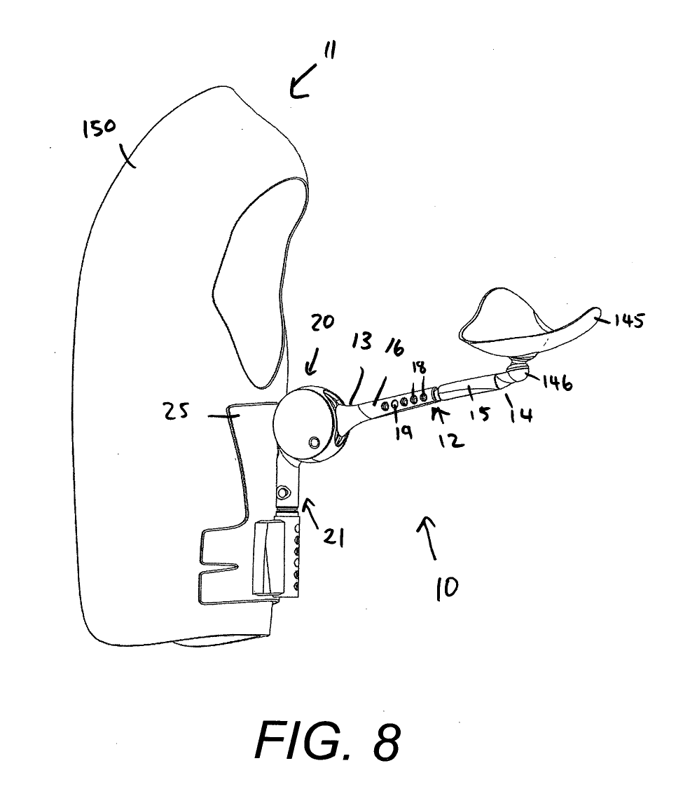

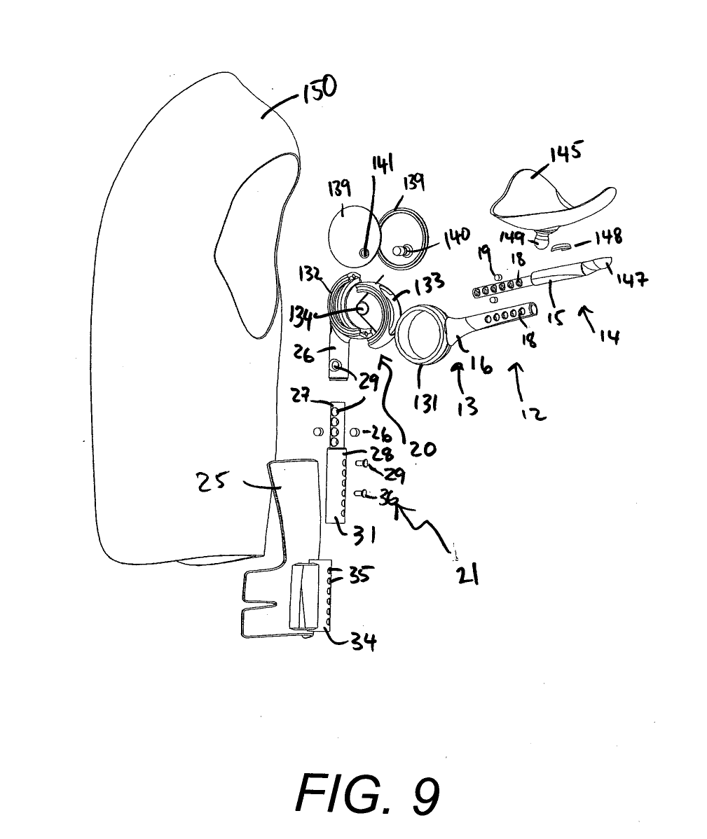

An alternative embodiment for adjusting the length of arm 12 is shown in FIGS. 8 and 9. Apertures 18 are provided in the first and second portions 15, 16 and a pin 19 enables length-fixing adjustment.

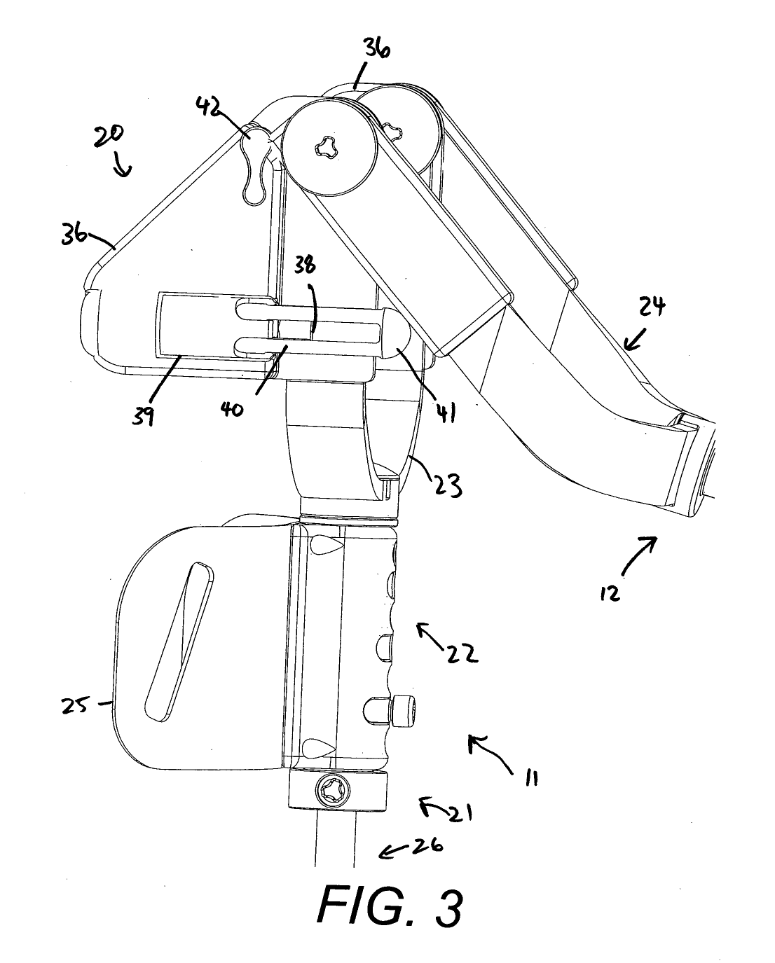

In FIGS. 1 to 7, the arm 12 is connected to the support 11, via a resilient joint 20. The resilient joint 20 is shown as attached to stem 21 of support 11. The support 11, at its first end 22, is connected to the resilient joint 20 by a first mounting bracket 23, and the arm 12 is connected to the resilient joint 20 at its first end 13, typically by a second mounting bracket 24, as shown in the FIGS. 1 to 7.

In FIGS. 1 to 9, the support 11 is shown in the form of torso plate(s) 25. Each torso plate 25 is curved so as to comfortably fit against a portion of the person's torso. Each torso plate 25 may be made of a flexible material to enable it to be adjusted to the shape of the person's torso.

The torso plates 25 are shown attached to stem 21. The stem 21 extends upwardly and comprises first and second portions 26, 27. The first and second portions 26, 27 are arranged to enable adjustment of the length of the stem 21 for each person using the shoulder brace 10. In FIGS. 1 to 3, the first portion 26 is sized to fit inside the second portion 27 (although in other embodiments, as shown in FIGS. 4-9, the first portion 26 can be sized to fit around the second portion 27).

In FIGS. 1 to 7, the first and second portions 26, 27 are telescopic, and are slidable with respect to each other. A locking mechanism 28, as shown in FIGS. 1, 2, 4 and 5, is used to lock the first and second portions 26, 27 at the stem length required for an individual. One such locking mechanism may include an adjustable screw nut. This allows a doctor, or the person, to unscrew the nut, move the first and second portions into the desired position, and tighten the screw, locking the first and second portions in position.

In an alternative embodiment, as shown in FIGS. 8 and 9, apertures 29 provided in the first and second portions 26, 27 and a pin 30 enable a length-fixing adjustment of the stem 21. The stem also comprises a third portion 31, also provided with apertures 32 and a further pin 33, which enables further adjustment of the length of the stem 21.

The plate 25 also comprises a holder 34 for receiving the stem 21, in particular the third portion 31 of the stem 21. The holder 34 comprises a tube having apertures 35 formed along its length. Another pin 36 may be used to selectively engage the apertures 35 in the holder 34 and the apertures 35 in the third portion 31 of the stem 21, enabling the effective length of the stem 21 to be further adjusted as required.

The resilient bias of the arm 12 is provided by a resilient joint 20 acting between the arm 12 and the stem 23. The resilient joint 20 can be provided in a number of different embodiments, as shown in FIGS. 1, 4, 6 and 8.

As shown in FIGS. 1 to 3, the resilient bias of the arm 12 is provided by resilient joint 20. The resilient joint 20 comprises two spaced, opposing joints 36 and 37. The two opposing joints 36 and 37 are mirror images of each other, and as such, only joint 36 will be described herein.

Joint 36 comprises a so-called resistance mechanism that comprises a compression spring 38 encased in telescopic tubes 39 and 40. In this embodiment the resistance mechanism is in a horizontal position, although it will be apparent to a person skilled in the art that the position of this resistance mechanism may be altered to a differing angle with respect to the support 11 (e.g. 45° to the horizontal). The spring 38 can be adjusted by screwing the base of tube 39 into tube 40 to create greater resistance (i.e. a more compressed spring) and vice versa. The end of tube 40 acts against member 41, which is attached to second mounting bracket 24, to force arm 12 in an upward direction. A stop 42 is provided to minimise jarring that would otherwise occur when the arm 12 reaches it's highest and lowest positions.

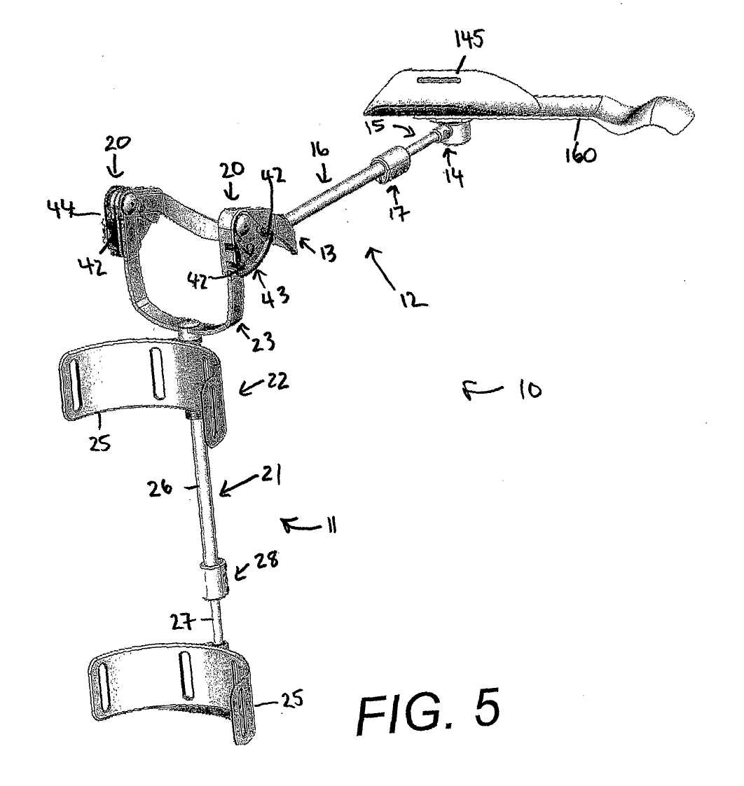

FIGS. 4 and 5 show an alternative resilient joint 20. This resilient joint 20 again comprises two spaced, opposing joints 43 and 44. The two opposing joints 43 and 44 are mirror images of each other, and again, only joint 43 will be described herein.

Joint 43 comprises two members 45 and 46, with a compression spring located between them. The spring forces member 46, which is attached to second mounting bracket 24, in an upward manner, to create the biasing effect on a person's arm. This joint also comprises stops 42 to minimise jarring when the arm 12 reaches its highest and lowest positions.

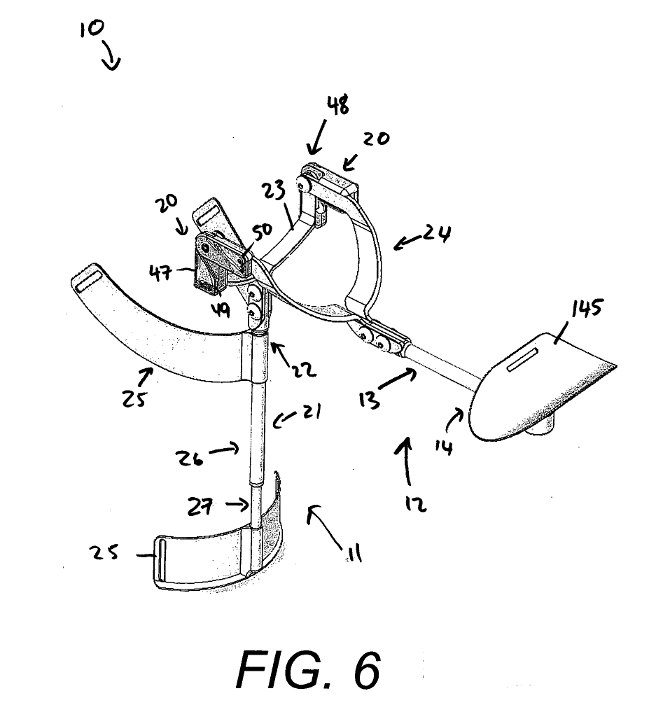

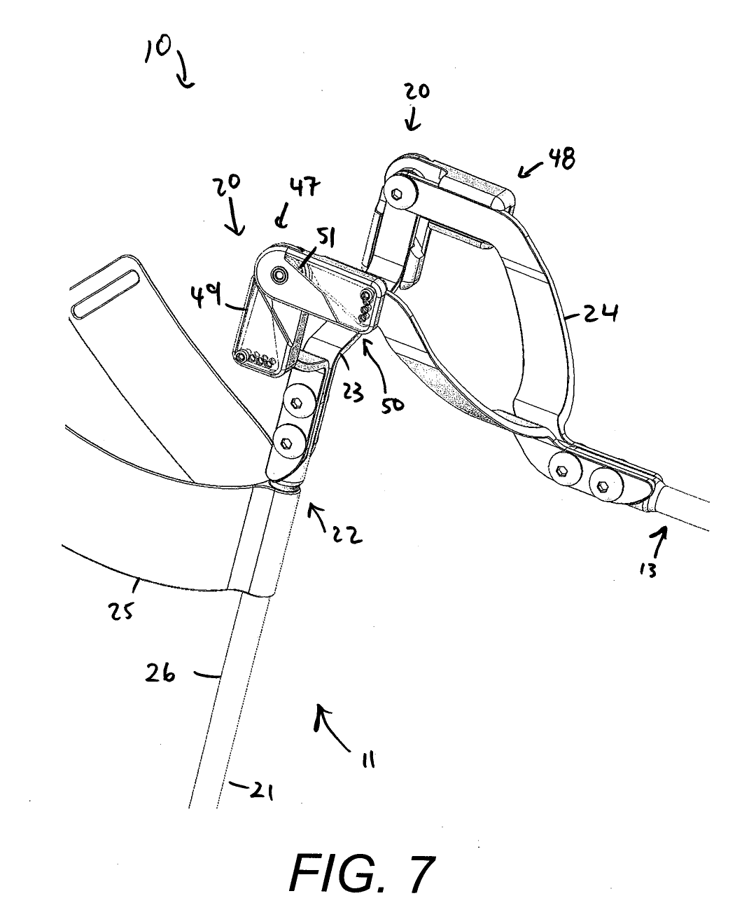

FIGS. 6 and 7 show a further alternative for resilient joint 20. This resilient joint 20 again comprises two spaced, opposing joints 47 and 48 and, because the joints 47 and 48 are mirror images of each other, again only joint 47 will be described herein.

Joint 47 comprises two members 49 and 50. Member 49 is stationary, and is attached to first mounting bracket 23 and member 50 pivots with respect to member 49, and is attached to second mounting bracket 50. A torsion spring 51 is encased between the two members 49, 50 to urge member 50 to pivot upwardly. This biased pivoting action creates the upward bias of arm 12, and creates the resistance for a person's arm.

FIG. 8 and, more particularly, FIG. 9 show yet another resilient joint 20 alternative. This resilient joint 20 comprises concentric first and second rings 131, 132. The first ring 131 is integrally formed with the arm 12 and the second ring 132 is integrally formed with the stem 21. The first ring 131 is shaped to fit inside the second ring 132 although, in other embodiments, the second ring may be shaped to fit inside the first ring. The second ring 132 may be split into two segments, as shown in FIG. 9, so as to enable the first ring 131 to be positioned in the second ring 132. A slot 133 is provided in the second ring 132 which enables the arm 12 to extend through the second ring 132 from the first ring 131. The slot 133 also allows for rotation of the first ring 131 inside the second ring 132, and this rotation is limited by the length of the slot 133, as the arm 12 will butt against the ends of the slot 133.

The resilient joint 20 also comprises a resistor which resists rotational movement of the first ring 131 inside the second ring 132. The resistor provides the resilient bias of the arm 12 and is shown in FIGS. 8 and 9 in the form of a spring 134. However, in a variation, the resistor is an elastic band. The spring 134 comprises two legs joined by a coil. One leg of the spring 134 is for engaging the first ring 131 and the other leg for engaging the second ring 132. The first and second rings 131, 132 each have a respective aperture for receiving a leg of the spring 134. The legs are retained in the apertures by the depth of the apertures.

It is noted that although the first ring aperture may be axially aligned with the length of the arm 12, it can be positioned anywhere in the first ring 131. The spring 134 is positioned inside both the first and second rings 131, 132. The first ring 131 also has a slot formed therein, which enables one of the legs of the spring 134 to extend through the first ring 131 (through the slot) into the second ring aperture.

With the legs of the spring 134 positioned in the apertures, any rotation of the arm 12 causes compression of the spring 134, in particular compression of the coil of spring 134, against the resilient bias of the spring 134. Thus, the further the arm 12 is rotated downwards, the greater the resistance to further movement of the arm 12 in that direction.

The legs of the spring 134 can be readily moved into and out of the apertures. Thus, one spring can be easily replaced with another spring. This enables a desirable treatment process during rehabilitation of a person's shoulder, whereby springs of increasing resilience can be positioned in the resilient joint 20 of the shoulder brace 10 over time as the person's muscles, ligaments and tendons strengthen.

The resilient joint 20 also comprises caps 139 to be positioned either side of the concentric rings 131, 132 to close over the sides of the rings 131, 132. One of the caps has a spigot 140 formed on its inner surface for engaging with an aperture 141 in the other cap to lock the caps in position either side of the rings 131, 132. The caps 139 can be readily removed from the sides of the rings 131, 132, in particular to replace the spring 134 inside, by pushing the spigot 140 out of the aperture 141.

The first ring 131 has a groove formed around its outer surface. The second ring 132 has a ridge formed around its inner surface for engaging the groove in the outer surface of the first ring 131. The ridge thus acts as a track for the first ring 131 to move along as it rotates, providing for controlled movement of the first ring 131 with respect to the second ring 132 and hence controlled movement of the arm 12 with respect to the support 11. This controlled movement of the arm 12 facilitates controlled movement of the person's recovering muscles, ligaments and tendons and hence is more conducive to proper healing of the injured or post-operated shoulder.

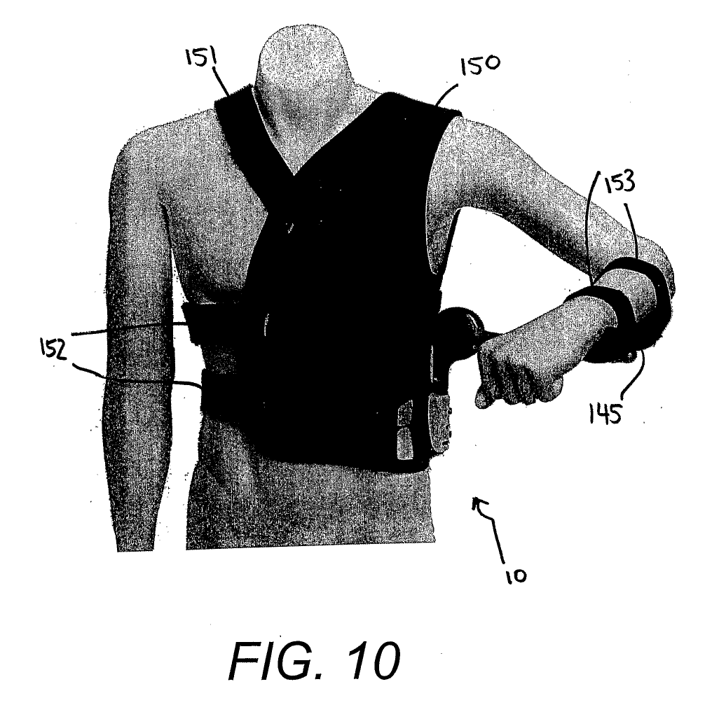

As shown in the Figures, the shoulder brace 10 also comprises a cradle 145 for cradling the person's forearm. The cradle 145 is ergonomically shaped to comfortably fit the person's forearm. The cradle 145 may be made of a flexible material which can be readily shaped to the person's forearm. The cradle 145 is connected to the second end 14 of the arm 12 by a ball and socket joint. This type of joint allows freedom of movement in 360° and thus allows for some limited, yet controlled, movement of the person's arm. The socket of the joint is formed by a cup 147 a U-shaped clip 148 which fits over the ball 149 when it is positioned in the cup 147. This prevents the ball 149 lifting out of the cup 147 and the joint being broken. Forearm straps 153 are also provided (see FIG. 10), connected to the cradle 145 to keep the person's arm strapped in the cradle 145.

In FIGS. 4 and 5 an additional wrist support extension is shown. It can be connected to arm 12 between cradle 145 and arm second end 14. It is designed to ergonomically support a person's wrist, whilst still allowing movement of the wrist.

In FIGS. 8 to 10, the support 11 is shown further comprising a vest portion 150 to be worn over the person's torso, adjacent to the shoulder being supported by the shoulder brace 10. The vest portion 150 is preferably made of a soft material which can be readily adapted to the shape of the person's torso. The plate 25 is connected to the vest portion 150. While not shown in FIGS. 1 to 7, the vest portion 150 can be used in a similar manner as shown in FIGS. 8 to 10. The plate(s) 25 can also be attached to vest portion. The shoulder brace 10 also comprises an opposing shoulder strap 151 for fitting over the person's opposing shoulder and two torso straps 152 (although the shoulder brace 10 may have only one such torso strap) for fitting around the opposing side of the person's torso and attaching to the vest portion 150. The opposing shoulder strap 151 and torso straps 152 are connected to the vest portion 150.

In the claim which follows and in the preceding description, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments.

1-26. (canceled)

27. A shoulder brace for supporting a person's injured or post-operated shoulder comprising:
a support for the person to wear; and
an arm connected to the support by spaced, opposing joints located, in use, anteriorly and posteriorly on the person, the arm being adapted for supporting the person's arm in a raised position, wherein the arm is movable with respect to the support and is resiliently biased against downward movement with respect to the support.

28. A shoulder brace as claimed in claim 27 wherein the arm comprises first and second portions to enable adjustment of the length of the arm.

29. A shoulder brace as claimed in claim 27 wherein the support comprises at least one plate.

30. A shoulder brace as claimed in claim 29 wherein the at least one plate is shaped to fit comfortably against a person's torso.

31. A shoulder brace as claimed in claim 29 comprising at least two plates and a stem interconnecting the at least two plates.

32. A shoulder brace as claimed in claim 31 wherein the joints connect the arm and the stem and are adapted to provide the resilient bias against downward movement.

33. A shoulder brace as claimed in claim 31 wherein each joint comprises a resistor which resists movement of the arm.

34. A shoulder brace as claimed in claim 33 wherein the resistance of the resistor can be adjusted.

35. A shoulder brace as claimed in claim 33 wherein each joint can be removably replaced with a joint of differing resistance.

36. A shoulder brace as claimed in claim 33 wherein each joint comprises a first member which in use sits in a generally static location with respect to a person's shoulder, and a second member pivotally mounted to the first member, the second member being adapted for coupling to the arm.

37. A shoulder brace as claimed in claim 36 wherein the resistor is a spring located for operation between the first and second members.

38. A shoulder brace as claimed in claim 36 wherein, in each joint, the first member is connected to the stem by a first mounting bracket, and the second member is connected to the arm by a second mounting bracket.

39. A shoulder brace as claimed in claim 38 wherein the second mounting bracket is shaped to support the person's arm.

40. A shoulder brace as claimed in claim 27 wherein the arm further comprises a cradle located for cradling the person's forearm in use.

41. A shoulder brace as claimed in claim 40 further comprising an extension support attachable to the cradle, for supporting the person's wrist, in use.

42. A shoulder brace as claimed in claim 27 wherein the support further comprises at least two straps for attaching the support around and to the person's body.

43. A shoulder brace as claimed in claim 42 wherein a first strap is attached over the person's non-injured shoulder and subsequent strap(s) are attached around the person's torso.

44. A shoulder brace as claimed in claim 27 wherein the support further comprises a vest portion to be worn over the persons torso adjacent to the shoulder being supported by the shoulder brace.

45. A method of treating a person's injured or post-operated shoulder, the method comprising the steps of:
(a) wearing the shoulder brace as claimed in any one of the preceding claims; and
(b) having the person use their arm, which has the injured or post-operated shoulder, to apply a force against the resilient bias of the arm of the shoulder brace.

46. A method as claimed in claim 45, wherein the person periodically applies a force against the resilient bias of the shoulder brace's arm during treatment.

47. A method as claimed in claim 45, further comprising the step of increasing the strength of the resilient bias of the arm of the shoulder brace.

48. A method as claimed in claims 47 wherein the step of increasing the strength of the resilient bias occurs in stepwise increases over the length of treatment.

49. A method as claimed in claim 47 wherein the step of increasing the strength of the resilient bias comprises replacing the resistor which resists movement of the arm with a resistor of greater resistance.

50. A method as claimed in claim 49 wherein replacing the resistor comprises replacing the spring inside the resilient joint.

51. A method as claimed in claim 45 wherein the treatment occurs for approximately 12 to 18 weeks.
